(19) United States
(12) Patent Application Publication
(10) Pub. No.: US 2020/0069406 A1
(43) Pub. Date: Mar. 5, 2020

(54) METHOD AND SYSTEM FOR DETERMINING CALVING INFORMATION

(71) Applicant: Nedap N.V., Groenlo (NL)

(72) Inventors: Jeroen Martin Van Dijk, Enschede (NL); Rudie Jan Hendrik Lammers, Eibergen (NL)

(73) Assignee: Nedap N.V., Groenlo (NL)

(21) Appl. No.: 16/614,498

(22) PCT Filed: May 17, 2018
(86) PCT No.: PCT/NL2018/050327

§ 371 Date: Nov. 18, 2019

(30) Foreign Application Priority Data
May 18, 2017 (NL) 2018938

(51) Int. Cl. A61D 17/00; A61B 5/0245; A61B 5/021; A61B 7/00; A61B 5/00; G08B 21/18

(52) U.S. Cl. CPC A61D 17/008; A61B 5/02455; A61B 5/021; A61B 7/00; A61B 2503/40; A61B 5/0022; A61B 5/6816; A61B 5/6871; G08B 21/182; A61B 5/0077

(57) ABSTRACT

Method and system for determining calving information about a moment at which a cow is going to calve, a period in which a cow is going to calve or the moment at which the cow has calved. The system comprises a sensor and a signal processing unit which is configured to process signals obtained with the sensor. With the sensor a heartbeat of the cow is measured so that the signals obtained with the sensor comprise information about the measured heartbeat of the animal. With the signal processing unit an increased heartbeat is recognized according to a predetermined criterion on the basis of signals obtained with the sensor. If the increased heartbeat is recognized with the signal processing unit, the signal processing unit generates an alarm signal which comprises the calving information.

### BACKGROUND OF THE INVENTION

The invention relates to a method and system for determining calving information of a cow. By ‘calving’, giving birth to a calf is meant.

Calving information is very important for dairy cows. Dairy cows in calf are generally milked until about two months prior to calving, after that a milking stop takes place. By milking being stopped, the cow is enabled to build up a good condition for giving birth to the calf. Due to milking being discontinued, the cow's hormone regulation changes. As a consequence, physiological parameters, e.g., blood pressure and heartbeat, may become significantly different with respect to a cow that is milked. When in an advanced stage of pregnancy, the cow is inspected several times by the farmer or a veterinarian. Based on the loss of appetite of the cow, thickening or edema formation in the udder, possibly together with a fall in body temperature, it can be inferred that calving is imminent. That is, calving will begin within a day. The cow also needs to be separated from other animals in a timely manner before the actual calving in view of stress and hygiene.

A problem of the currently prevailing method is that it is time intensive. Also, with this method, it is established that calving will take place by a veterinarian or farmer himself only shortly before calving, that is, in most cases, when calving is imminent. Further, the farmer will have to remain ready until the cow actually goes into labor, the reason for this being that calving proceeds more successfully under the close supervision of the farmer or veterinarian. Such readiness, or also vigilance, is extremely inconvenient for farmers. It often happens, as a consequence, that the farmer is hindered from meeting his obligations elsewhere or cannot enjoy a good night's sleep. Furthermore, untimely separation, be it too early or too late, of the cow from other cows will lead to stress. Stress in the cow generally has adverse consequences for the unborn calf.

In accordance with these circumstances, there is a need to relieve both farmer and cow in the approach to calving and throughout calving itself. In consequence, there is also a need for the accurate provision of information about the moment or the period of calving through to the moment at which the cow has calved.

It is an object of the present invention to provide a system and a less labor-intensive method for timely determining a moment at which, or period in which, the cow is going to calve or the moment at which the cow has calved. It is therefore an object of the invention to eliminate at least one of the existing disadvantages of the current method or reduce the consequences thereof. It is also an object of the present invention to provide ameliorated or alternative solutions which can be implemented in a simpler manner and which, moreover, can be made comparatively inexpensively. Alternatively, it is an object of the invention to provide to the public an at least useful option.

### SUMMARY OF THE INVENTION

To this end, the invention provides inter alia a method for determining calving information about a moment at which a cow is going to calve and/or a period in which a cow is going to calve, and/or the moment at which the cow has calved. In the method, use is made of a sensor and a signal processing unit. The signal processing unit is configured to process signals obtained with the sensor. With the aid of the sensor, a heartbeat of the cow is measured, so that the signals obtained with the sensor comprise information about the measured heartbeat of the animal. Heartbeat is understood to mean the frequency of the beating of the heart. By the signal processing unit, an increased heartbeat is recognized according to at least one predetermined criterion on the basis of signals obtained with the sensor. If the increased heartbeat is recognized with the signal processing unit, the signal processing unit generates an alarm signal which comprises the above-mentioned calving information. Thus, the farmer can arrange for himself to be informed by the alarm signal about the calving without him needing to be vigilant continuously. This method has the advantage of serving to reassure the farmer so that he knows, for example, that the moment, or the period, of calving is happening, or is about to happen, preferably within a predetermined period. The farmer can then infer from this whether action or no action on his part is required.

Optionally, the signal processing unit is configured to recognize that the measured heartbeat is greater than a first predetermined value. An advantage of this is that the predetermined value can be chosen such that the farmer can be informed with high accuracy about a status of the cow in respect of the expected upcoming calving and, more important still, that it can be indicated when calving begins, is in progress, or is over.

Optionally, the first predetermined value is equated to an average heartbeat of a cow or cows over a predetermined period in the past plus a first increment. An advantage of this is that in this way the physiological changes that influence the heartbeat in the approach to calving are used for more accurately determining when calving begins, is in progress, or is over. Further, the predetermined period may also be in the milking stop period. An advantage is that the influences of hormonal changes due to the milking stop are thus taken into account, so that the value of the average heartbeat over a part of this period means a more accurate information for the farmer.

Optionally, the first predetermined value is determined with the aid of the sensor and the signal processing unit. The advantage is that this determination is directed to one specific animal. Accordingly, the mutual variation among animals is overcome. This enhances the accuracy of the information which is inherent to the alarm signal. An example is that the predetermined value is an average heartbeat of the cow over a few days or weeks during a period of milking stop and during gestation. It is pointed out here that the average heartbeat during the period of milk stop takes on a new average value which is different than during milking and outside of gestation due to hormonal influences. This new average value, in turn, is different than the value a few days and minutes before calving. The extent to which the heartbeat changes to achieve the new value may be different for each animal due to various factors, such as stress, age, breed and weight.

Optionally, the first predetermined value is determined for a group of animals and inputted at the signal processing unit. The advantage here is that this measurement is once-only and can be applied to several animals, also a multiplicity of cows. This saves the operation of determining the predetermined value for each individual cow.

Optionally, the signal processing unit is configured to recognize that the measured heartbeat is greater than a second predetermined value which is greater than the first predetermined value. Since the heartbeat varies during the day, and with various activities, it will be clear that the measured heartbeat can also be understood to be an average measured heartbeat. To the skilled person, it will be clear over what period the average measured heartbeat is an average to exclude incidental heartbeat increases resulting from short-lived activities. The advantage of this is that the operation of comparing heartbeat is thus no longer labor intensive, and human misinterpretation, for example resulting from short-lived heartbeat increase, and false alarm, is prevented.

Optionally, the second predetermined value is equated to the first predetermined value plus an increment. An advantage is that the second value consequently depends on the first predetermined value, so that it can be determined for an individual animal or a group of animals and hence can be accurate and animal-specific, while saving a further measurement.

Optionally, the signal processing unit is configured to generate a first alarm signal when the heartbeat is greater than the first predetermined value. The first signal indicates that the cow is going to calve within a predetermined first period. The time duration of the first period is predetermined. The advantage here is that this results in an expectation time, which the farmer can use to accurately make preparations around calving.

Optionally, the signal processing unit is configured to generate a second alarm signal when the heartbeat is greater than the second predetermined value. The second signal indicates that the cow is going to calve within a predetermined second period. Preferably, the second period is a very short time before calving. It is further preferred, when in combination with the preceding option, that the time duration of the first and second period is predetermined and the second period has a shorter time duration than the first period. The advantage is that this results in a two-step warning system, enabling more accurate anticipation and timely intervention. The two-step warning system in effect provides additional control and adjustment of initial prediction compared with a one-step warning system.

Optionally, the signal processing unit is configured to generate a third alarm signal when the cow has calved. This may be done by transmitting an alarm signal upon expiration of the second period. The advantage here is that the farmer is informed of the moment of calving, so that, also in case of absence, he can monitor that calving has taken place. In particular, the third alarm signal is generated with the aid of the signal processing unit when it is detected by the signal processing unit that the heartbeat, after having exceeded the first predetermined value, has fallen below the first predetermined value again and/or, for example, when it is detected by the signal processing unit that the heartbeat, after having exceeded the second predetermined value, has fallen below the second predetermined value again.

When the combination with the third alarm signal is made, there will result, for example, a three-step warning system which leads the farmer intuitively through the approach to the calving itself. With the three-step warning system, there occurs a double correction on initial predictions leading to parturition times. The system thus corrects itself and gives the farmer the time to correct himself along with these adjustments. The farmer can now always act in a timely manner, without being surprised.

Optionally, the animal is separated, also: segregated, from other animals when the first alarm signal is delivered. Because cows are social animals, a separation, when the animal is not ready for it, may be disadvantageous for a delivery. Often, in anticipation of delivery, cows are separated too early, or during routine examination it is realized that the cow should have been separated. It is advantageous to identify the moment of separation adequately in connection with hygiene and stress in approaching calving. With a system according to the invention, the first alarm can be accurately chosen to indicate to the farmer that, or when, separation is desirable. That way, separation does not need to be determined through labor-intensive physical or periodical monitoring of the animal.

Optionally, the animal is separated automatically when the first alarm signal is delivered. This may be done, for example, in that the alarm signal is sent to a barn management apparatus which manages the opening of barn stalls, or a gate. This may be done, for example, by means of mobile communication. After transmission of this information, a gate (or like barrier), such as that of the stall of the animal, can be closed to prevent the animal mingling with other animals. Accordingly, the advantage is that separation is carried out at the desired moment in the advanced pregnancy stage of down-calving while it simultaneously relieves the farmer.

Optionally, a signal is automatically sent to a farmer, for example to his mobile phone, when one of the first, second or third alarm signals are delivered. The advantage is that the farmer receives the alarm signals directly and thereby acquires knowledge about the status and progress of calving.

Optionally, the first period lasts 1-4 days. Preferably, the first period lasts 1.5-2.6 days. Together with or separately from the first period, the second period lasts 1-1440 minutes. Preferably, the second period, likewise together with or separately from the first period or preferred duration of the first period, lasts 5-60 minutes. An advantage is that the time periods correspond to a course of steps intuitive for a farmer in the approach to calving. As a result, preparatory actions for delivery can be undertaken in a timely and efficient manner.

Optionally, the sensor is a microphone, or a system including a first unit to transmit light and a second unit to receive light, or a blood pressure sensor, or a video camera. Where a system for transmitting and receiving light is concerned, such system may be configured for derivation of the heartbeat from reflection of transmitted light on the tissue and/or, at least partly, the transmission of light that after transmission propagates through the tissue of the animal, such as the cow. Preferably, the sensor is a microphone. It is further preferred that the heartbeat is measured with the sensor from the stomach of the cow. This has as an advantage that the heartbeat can be measured, without invasive methods, as closely as possible in the proximity of the heart. The sensor may also be attached to the ear. Furthermore, the sensor may also be integrated in a smart tag, or be attached to a smart tag. The smart tag may then be implemented as the ear stud known per se. The smart tag, and hence the sensor, can then be attached to the ear. In all cases, the sensor can then perform measurements on the tissue of or near the ear.

The sensor may also comprise a system including a first unit to send light to the tissue of the animal and a second unit to receive light from the tissue, while preferably the received light has propagated through the tissue of the animal and/or has reflected thereon. If the sensor is part of an ear stud, then, for example, the first unit may be part of a first part of the ear stud and the second unit may be part of a second part of the ear stud, while in use, in a known manner, the first and second part of the ear stud are respectively attached on either side of the ear.

According to a further aspect of the invention, there is provided, inter alia, a system for determining calving information about a moment at which a cow is going to calve and/or a period in which a cow is going to calve, and/or the moment at which the cow has calved. The system includes a sensor and a signal processing unit. The signal processing unit is configured to process signals measured with the sensor. The sensor is configured to measure a heartbeat of the cow so that the signals obtained with the sensor comprise information about the measured heartbeat of the animal. The signal processing unit is configured to recognize an increased heartbeat according to a predetermined criterion on the basis of the signals obtained with the sensor. The signal processing unit is configured for, if the increased heartbeat is recognized with the signal processing unit, generating an alarm signal which comprises the calving information mentioned.

Optionally, the signal processing unit is configured to recognize that the measured heartbeat is greater than a first predetermined value.

Optionally, the first predetermined value is equal to an average heartbeat of the cow over a predetermined period in the past plus a first increment.

Optionally, the system is so configured that the first value can be determined with the aid of the sensor and the signal processing unit. To this end, the signal processing unit could include means, such as an algorithm, to recognize heartbeats in signals which the sensor generates, to average these heartbeats over time and base the first value thereon. To this end, the signal processing unit could comprise a clock, such as a quartz clock, and a memory, such as a SRAM, DRAM, Z-RAM, A-RAM, ROM, PROM, EPROM, EEPROM or RAM, FeRAM, CBRAM, PRAM, SONOS, RRAM, racetrack memory, NRAM, 3D XPoint, and millipede memory. Correspondingly, this could be connected to an energy source such as a battery, for example a lithium-ion, zinc-carbon, alkaline, silver oxide, lithium-manganese oxide, mercury-zinc, both rechargeable and non-rechargeable, as well as an external energy source insofar as the system is not used internally.

Optionally, the signal processing unit is provided with the first predetermined value. The first predetermined value is determined for a group of animals.

Optionally, the signal processing unit is configured to recognize that the measured heartbeat is greater than a second predetermined value which is greater than the first predetermined value.

Optionally, the second predetermined value is equal to the first predetermined value plus a second increment.

Optionally, the signal processing unit is configured to generate a third alarm signal when the second period has expired or a heartbeat is measured that corresponds to the state of having calved, such as a fall in heartbeat after delivery, for example when it is detected that the heartbeat, after having risen substantially (for example by more than a predetermined percentage of an instantaneous heartbeat of the animal such as 10%, 20% or 30%), has fallen again. Optionally, the signal processing unit is configured to generate a first alarm signal when the heartbeat is greater than the first predetermined value and a second alarm signal when the heartbeat is greater than the second predetermined value. The first alarm signal indicates that the cow is going to calve within a predetermined first period and the second alarm signal indicates that the cow is going to calve within a predetermined second period. The time duration of the first and second period is predetermined and the second period has a shorter time duration than the first period. The third alarm signal may then be generated, for example, when it is detected by the signal processing unit that the heartbeat, after having exceeded the first predetermined value, has fallen below the first predetermined value again and/or, for example, when it is detected by the signal processing unit that the heartbeat, after having exceeded the second predetermined value, has fallen below the second predetermined value again. The third alarm signal can then indicate that the animal has meanwhile calved.

Optionally, the system is configured to separate the animal, for example, by opening a gate or like barrier so that the cow can enter a separation space, and closing the gate again when the first alarm signal is delivered. Also outside of this option, the processing unit may be implemented with an antenna for sending signals, and specifically all alarm signals mentioned, which can be captured by an apparatus controlling gates in the barn.

Optionally, the system is configured to separate the cow automatically, for example, by automatically opening a gate so that the cow can enter a separation space and automatically closing the gate again when the first alarm signal is delivered.

Optionally, the system is configured to automatically send a signal to a farmer, for example, to his mobile phone, when an alarm signal is delivered. This can be each of the first, second and third alarm signals.

Optionally, the first period has a predetermined length of 1-4 days, preferably 1.5-2.5 days and/or the second period has a predetermined length of 1-1440 minutes, preferably 5-60 minutes.

Optionally, the sensor is a microphone, a system including a first unit to send light and a second unit to receive light, a blood pressure sensor, or a video camera.

Optionally, the sensor is configured to communicate wirelessly with the signal processing unit, for example, via Bluetooth or a mobile network. The advantage here is that measurements on the cow can take place in a manner free of a physical connection and can be performed remotely.

Optionally, the sensor is included in a tag which can be worn by the animal, a bolus which can be included in one of the stomachs of the animal or in an implantable element. An advantage here is that measurement can thus be done close to the heart and that noise from outside the cow and due to ambient influences is reduced. As a result, measurements take place accurately. It will be clear that the bolus is so placed as not to be lost by regurgitation or vomiting. The bolus and the system are so designed that interaction with a possible magnet in any one of the stomachs of the cow is obviated. This can be done by manufacturing the system as much as possible using non-ferromagnetic alternatives for specific parts of the sensor and the signal processing unit, as well as any electrical connecting elements. This could also be done if a housing was implemented as a bolus.

Optionally, the sensor and the signal processing unit are included in a housing.

Optionally, the signal processing unit is configured to communicate wirelessly with a central computer to pass on an alarm signal generated by the signal processing unit to the central computer. Optionally, the central computer is a barn management apparatus or an apparatus as discussed earlier for controlling gates or like barriers in a barn or farm.

Optionally, the housing is implemented as a tag which can be worn by the animal, a bolus which can be included in the stomach of the animal or in an implantable element. Thus, the sensor is protected from ambient influences in the stomach or as an implant. Optionally, the signal processing unit is configured to generate a fourth alarm signal when in a third predetermined period which begins at the moment that the heartbeat has risen substantially (for example, by more than a predetermined percentage of an instantaneous heartbeat of the animal such as 10%, 20%, or 30%), it is not detected that the heartbeat, after having risen substantially (for example, by more than a predetermined percentage of an instantaneous heartbeat of the animal such as 10%, 20%, or 30%) has fallen again; and/or when in a third predetermined period which begins at the moment that the heartbeat has exceeded the first predetermined value, it is not detected by the signal processing unit that the heartbeat has fallen below the first predetermined value again and/or does not rise substantially further and/or when in a third predetermined period which begins at the moment that the heartbeat has exceeded the second predetermined value, it is not detected by the signal processing unit that the heartbeat has fallen below the second predetermined value again and/or does not rise substantially further. The third period is a period of time which may be arbitrarily chosen by the skilled person to be informed of a persistent increased heartbeat which exceeds the endpoint of this period. The third period can then begin when with the signal processing unit the increased heartbeat is recognized.

Optionally, the third period begins when with the signal processing unit the increased heartbeat is recognized according to the at least one predetermined criterion, the third period of time extending to beyond the first period and/or the second period and/or beyond a period where calving is becoming endangered. The skilled person will know that calving is not going well when the detected increased heartbeat persists too long. An advantage is that the farmer is informed about a deviant course of calving with increased risk of complications.

### DESCRIPTION OF THE DRAWINGS

The invention will be further clarified by the description of a few specific embodiments, using references to the appended figures. The detailed description provides examples of possible modes of use of the invention. These modes of use should not be regarded as the only possible embodiments that fall within the purview of the invention. The scope of the invention is defined in the claims, and the description should be regarded as being illustrative without thereby being limiting on the invention.

FIG. 1 shows a schematic method for determining calving information about a moment at which a cow is going to calve and a period in which a cow is going to calve;

FIG. 2 schematically shows a system for determining calving information about a moment at which a cow is going to calve and a period in which a cow is going to calve;

FIG. 3 schematically shows an application of the system in a cow; and

FIG. 4 schematically shows an application of the system in a cow, where the sensor is part of a smart tag.

### DETAILED DESCRIPTION OF THE INVENTION

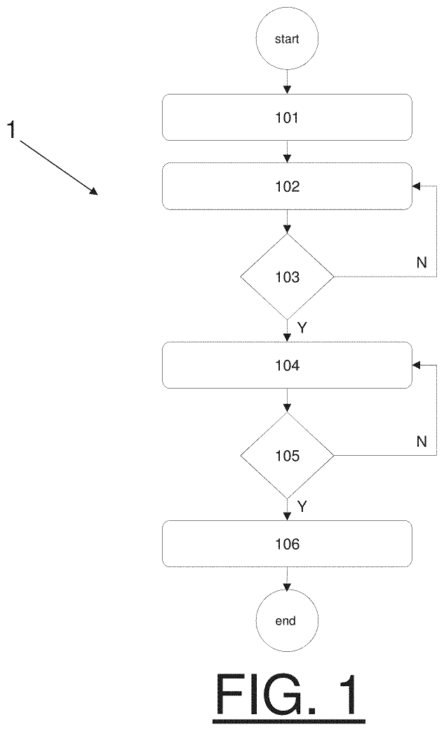

FIG. 1 shows a schematic method 1 for determining information about a first and a second period within which calving may be expected.

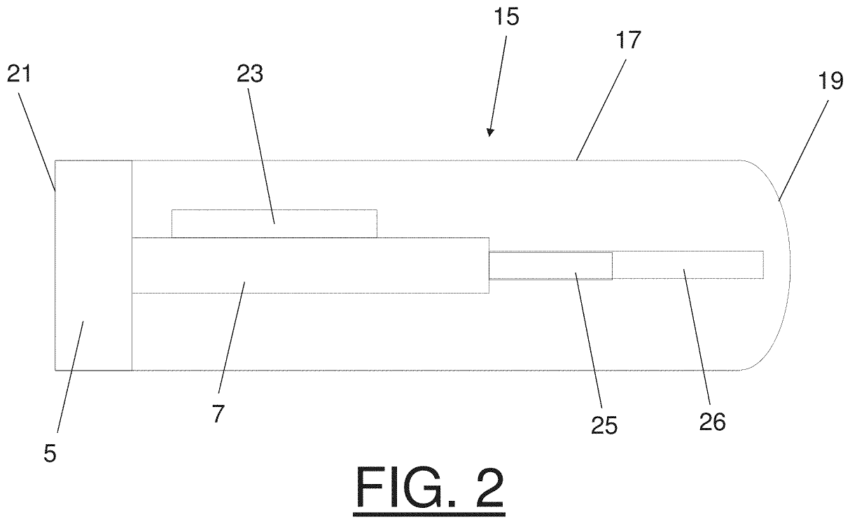

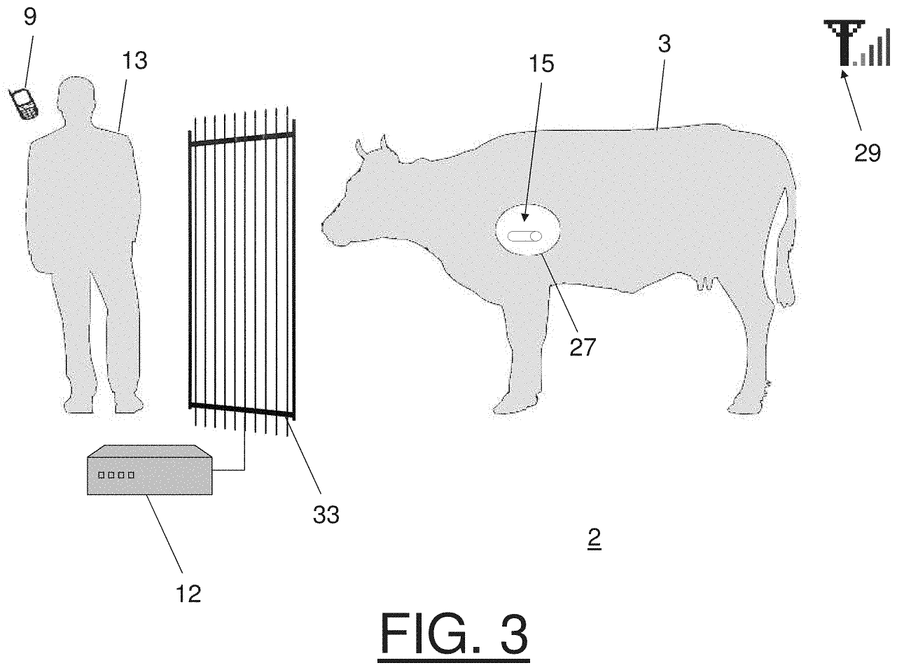

In a first step 101, sound of the heart of a cow 3, represented in FIG. 3, is measured with a sensor 5, here a microphone, represented in FIG. 2. The microphone converts the measured sound into a signal. The first step 101 leads to a second step 102.

In the second step 102 the signal from the sensor is obtained by a signal processing unit 7, represented in FIG. 2. The signal processing unit 7 processes this signal and, on the basis of the signal over time, determines a heartbeat frequency, also the heartbeat. The second step 102 leads to a third step 103.

In the third step 103, the signal processing unit 7 compares the heartbeat with a first predetermined value. The first predetermined value in this example is an average heartbeat of the cow 3 in the past during the milking stop two days before calving. The first predetermined value can be, for example, 79-85 beats per minute. When the heartbeat is higher than the first predetermined value, the signal processing unit 7 recognizes an increased heartbeat and the third step 103 leads to a fourth step 104. When this is not the case, the third step 103 leads back to the second step 102.

In the fourth step 104, the signal processing unit 7 generates a first alarm signal. This first alarm signal is sent by the signal processor 7 to a mobile phone 9, represented in FIG. 3, via a transmitter 25 and an antenna 26, represented in FIG. 2. Via the mobile phone 9, the farmer 13 is informed in this step that the first period of, for example, two days has begun, the expectation being that within this first period calving will take place. More generally, the first period has a predetermined length of 1-4 days, preferably 1.5-2.5 days.

In this step, the first alarm signal is also sent to the central computer 12 for closing a gate 33 to separate the cow 3 from other cows (not shown, but conventional). The fourth step 104 leads to a fifth step 105.

In the fifth step 105, the signal processing unit 7 compares the heartbeat with a second predetermined value. It is equal to the first predetermined value plus an increment. The increment is animal-specific and in this example has been chosen such that the second predetermined value corresponds to a peaking heartbeat of the cow one hour prior to calving. The second predetermined value can be, for example, 100-120 beats per minute. When the heartbeat is higher than the second predetermined value, the signal processing unit 7 again recognizes an increased heartbeat and the fifth step 105 leads to a sixth step 106. When this is not the case, the fifth step 105 leads back to the second step 102. The first alarm signal in this example is sent once only.

In the sixth step 106, the signal processing unit 7 generates a second alarm signal. This second alarm signal is also sent by the signal processing unit 7 with the aid of the transmitter 25 and the antenna 26 to the mobile phone 9. Via the mobile phone 9 the farmer 13 is informed in this step that the second period has started, the expectation being that the animal will calve within the second period. The second period in this example has a predetermined length of one hour. More generally, the second period has a predetermined length of 1-1440 minutes, preferably 5-60 minutes.

It will be clear that the steps as described above do not necessarily need to take place in the same order. Also, other steps may take place. Thus, by the signal processing unit 7 and with the aid of the transmitter 25 and the antenna 26, a third alarm signal may be generated and transmitted when it is detected that the heartbeat after having risen substantially (for example by more than a predetermined percentage of an instantaneous heartbeat of the animal such as 10%, 20% or 30%) has fallen again. Also, for example, the third alarm signal may be generated when it is detected by the signal processing unit that the heartbeat after having exceeded the first predetermined value has fallen below the first predetermined value again and/or, for example, when it is detected by the signal processing unit that the heartbeat after having exceeded the second predetermined value has fallen below the second predetermined value again. The third alarm signal can then be indicative of calving having taken place.

FIG. 2 schematically shows a unit 15 for determining information about the first and second period of calving, especially when the periods begin. The unit 15 comprises a housing 17. The housing in this example is implemented as a bolus, in that it has a convex front 19 and a flat rear 21 for simple physical guidance to and into the stomach. The housing 17 is watertight, is of rigid implementation and made from a nontoxic plastic, such as PET, for protecting components in the unit 15 from gastric juices and constraint by food and entrails of the cow 3. At the rear 21, within the housing 17, the unit 15 is provided with a microphone 5. The microphone 5 is communicatively connected with the signal processing unit 7. Captured sounds are converted into signals which are received and processed by the signal processing unit 7. The unit 15 is for instance provided with a battery/accumulator 23 for providing energy to the signal processing unit 7 and the transmitter 25 for carrying out the method steps of FIG. 1. Quite apart from this example, it is also possible to provide the unit 15 as a smart tag. The sensor is then attached to the smart tag or integrated with the smart tag. Instead of a microphone 5, any other earlier-mentioned sensor could also be used then. Instead of internal measurements in the stomach, measurements on other parts of the animal, such as the ear, could take place.

FIG. 3 schematically shows the application of the unit 15 in the cow 3 in a system 2. In this example, the unit of FIG. 2 is included in the paunch 27 of the cow 3. Optionally, also apart from this example, this can also be any one of the other stomachs of the cow 3. Here, the processing unit 7 transmits with the aid of the transmitter 25 and antenna 26 a first alarm signal via a mobile network 29 to the mobile phone 9 of the farmer 13 and to a central computer 12 of the system. The central computer is configured for controlling the gate 33, or like barrier, of a separation space (not represented, but conventional). The central computer 12 is configured to close the gate 33 after receipt of the first alarm signal. In this example, the separation space is a hygienic stall for down-calving cows. The farmer 13 is thus informed via the first warning signal that calving will take place within two days and that the cow 3 is separated. Further, the processing unit 7 sends via the transmitting device 25 and the antenna 27 the second alarm signal via the mobile network 29, this time merely to the mobile phone 9 of the farmer 13. Thus, the farmer is informed that calving will take place within an hour.

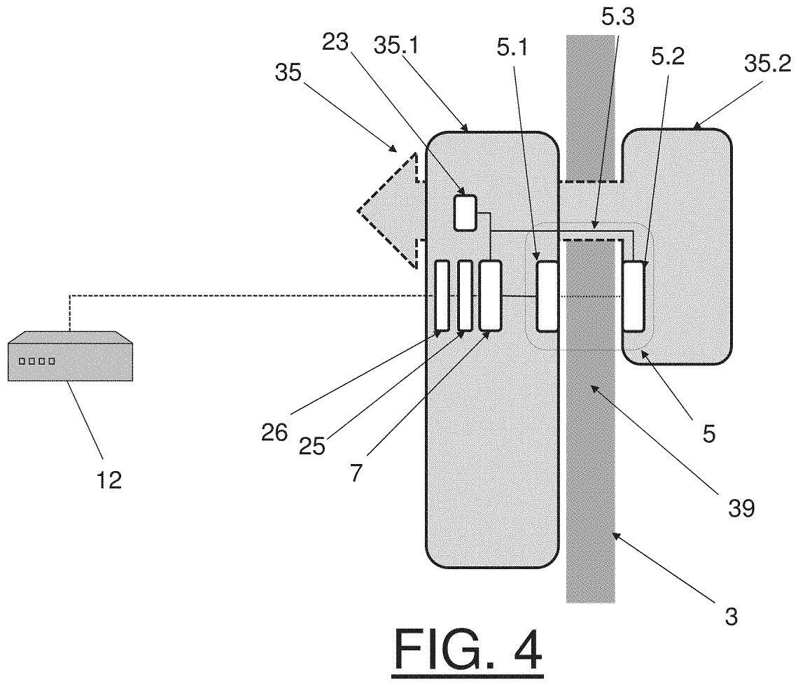

FIG. 4 schematically shows the application of the unit 15 where the sensor 5/unit 15 is part of a smart tag, in this example a smart tag which is implemented as an ear stud 35 which, in use, is attached to the ear of the cow 3. The ear stud 35 has a first part 35.1 and a second part 35.2 which in a manner known per se are coupled to each other through the ear and constitute the housing 17 of the sensor 5. As a result of this coupling the first and second parts 35.1, 35.2 of the ear stud 35 are respectively attached on either side of the ear of the cow 3. The smart tag 35 is configured to be able to communicate wirelessly with the central computer 12 and/or the mobile phone 9. To that end, the first part 35.1 of the ear stud is provided with the transmitter 25 and the antenna 26, functionally comparable to the transmitter device 25 and antenna 26 of FIG. 2. The sensor 5 comprises a first unit 5.1 to send light to the ear tissue 39 of the cow 3 and a second unit 5.2 to receive light from the ear tissue 39. In this example, the first unit 5.1 comprises to that end a light source, such as an LED. The second unit 5.2 comprises to that end a light sensor. The ear tissue 39 is in-between the first part 35.1 and the second part 35.2 of the ear stud 35, so that light from other sources cannot influence the measurement. In this example, the received light has propagated through the ear tissue 39 of the cow 3. In this example, the first unit 5.1, the signal processing means 7, the transmitter 25 and the antenna 26 are included in the first part 35.1 of the ear stud and the second unit 5.2 is included in the second part 35.2 of the ear stud 35. The first 35.1 and second part 35.2 of the ear stud 35 are electrically and communicatively connected with each other. This can be done, for example, by means of an electrical connection 5.3 with which information is transferred from the second unit 5.2 to the signal processing unit 7, and electrical energy is transferred from the first part 35.1 to the second part 35.2 for the operation of the second unit which comprises the light sensor. The signals generated by the second unit comprise information about the heartbeat of the animal in that the blood pressure and hence the light transmissivity of the ear varies depending on the heartbeat. These signals are then fed to the signal processing unit 7 for further processing as discussed above. Alarm signals can then be transmitted with the transmitter 25 to the mobile phone and/or the central computer as discussed above if the signal processing unit recognizes an increased heartbeat from the signals received from the sensor. Alternatively, the unit 35 is implemented as a measuring unit according to NL2015582, in particular according to FIG. 7 of NL2015582. The light sensor is then implemented as a frequency meter for heartbeat. Alternatively, the first unit 5.1 and the second unit 5.2 may also be part of a common part of the smart tag or ear stud 35. The first unit 5.1 and the second unit 5.2 of the sensor 5 may then be set up, respectively, to reflect light on the tissue and to measure light reflected on the tissue. Alternatively, it holds for each embodiment that the signals that have been obtained with the sensor 5 can also be supplied with the transmitter 25 and antenna 26 to the central computer 12 for processing with the computer 12 as indicated above for the signal processing unit 7. The central computer then comprises the signal processing unit and can then generate the alarm signals and send them to the mobile phone 9 and/or control the separation gate 33 as discussed above.

Thus described are a method and system for determining calving information about a moment at which a cow is going to calve, a period in which a cow is going to calve or the moment at which the cow has calved. ‘Going to calve’ should be understood to mean the moment at which calving begins. The system comprises a sensor and a signal processing unit which is configured to process the signals obtained with the sensor. With the sensor, a heartbeat of the cow is measured so that the signals obtained with the sensor comprise information about the measured heartbeat of the animal. With the signal processing unit, an increased heartbeat is recognized according to a predetermined criterion on the basis of the signals obtained with the sensor. If the increased heartbeat is recognized with the signal processing unit, the signal processing unit generates an alarm signal which comprises the above-mentioned calving information.

In particular, it is furthermore possible, in each embodiment, for the signal processing unit to be configured to generate a fourth alarm signal when in a third period it is not detected that the heartbeat, after having risen substantially (for example by more than a predetermined percentage of an instantaneous heartbeat of the animal such as 10%, 20% or 30%), has fallen again. The third period can then start at the moment that the heartbeat has risen substantially (for example by more than a predetermined percentage of an instantaneous heartbeat of the animal such as 10%, 20% or 30%). This third period can extend, for example, from A1-A2, more particularly from A3-A4, still more particularly from A5-A6. Here, A1, A3 and A5, respectively, are possible times at which the third period begins. A2, A4 and A6, respectively, are possible times at which the third period ends. Times A1, A3 and A5 are, in particular, moments at which the heartbeat has (first) gotten substantially increased relative to the first predetermined value. The first predetermined value is, for example, 79-85 beats per minute. A1 may for instance have been chosen so as to correspond to the time at which nesting behavior starts. The beginning of nesting behavior can be recognized by a heartbeat that rises at least beyond 89-93 beats per minute. Time A2 can then, for example, be chosen such that A1-A2 has a length of 40 minutes to 7 hours, more particularly, 6 hours. A3 may, for example, be chosen such that it corresponds to the time at which the allantois becomes visible. The allantois becoming visible can be recognized by a heartbeat which rises at least beyond 97-103 beats per minute. Time A4 may then, for example, be chosen such that A3-A4 has a length of one hour. Time A5 may, for example, be chosen such that it corresponds to the start of the last phase of calving where a part of the still unborn calf is already visible. The last phase of calving can be recognized by a heartbeat that peaks at at least 105-110 beats per minute. Time A6 may then, for example, be chosen such that A5-A6 has a length of 15-45 minutes, more particularly, 30 minutes. Times A1, A3 and A5 are successive moments in time. An increased heartbeat, and hence the beginning of the third period, may further have been recognized already according to the at least one predetermined criterion, prior to points of time A1, A3 and A5. Additionally or alternatively, the fourth alarm signal may also be generated by the signal processing unit when in a third predetermined period it is not detected by the signal processing unit that the heartbeat, after having exceeded the first predetermined value, has fallen below the first predetermined value again (this is indicative of calving not having taken place yet) and/or does not rise substantially further (this indicates that calving has not started yet). The first predetermined value can be a heartbeat of 79-85 beats per minute. In particular, the third period then begins when the first predetermined value is exceeded. The length of the third period is predetermined and has, for example, a value of 36 hours. Optionally, the third time period extends to beyond the first period by, for example, a maximum of 7 hours. Additionally or alternatively, the fourth alarm signal may also be generated by the signal processing unit when in a third predetermined period it is not detected by the signal processing unit that the heartbeat after having exceeded the second predetermined value has fallen below the second predetermined value again and/or does not rise substantially further. The second predetermined value can be the first predetermined value with an increment. The increment may be chosen, for example, between 2 and +15 beats per minute, more particularly between 5 and 10 beats per minute. In particular, the third period then begins when the heartbeat has exceeded the second predetermined value. The length of the third period is predetermined and has, for example, a value of 10 hours. Optionally, the third time period extends to beyond the second period by, for example, 1-7 hours.

On the basis of the fourth alarm, it can be established that a delivery is not going well. Other variants to detect a heartbeat persisting too long are also possible. For the sake of clarity and conciseness of the description, features have been described here as part of the same or of separate embodiments. It will be clear to those skilled in the art that embodiments comprising combinations of any or all of the described features also fall within the scope of protection of the invention. Within the purview of the skilled person, modifications are possible that are understood to be within the scope of protection. Further, all kinematic inversions are understood to be within the scope of protection of the present invention. Expressions such as “consisting of”, when used in this description or the appended claims, should be construed not as an exhaustive enumeration but rather in an inclusive sense of “at least consisting of”. Designations such as “a” or “one” may not be construed as a limitation to just a single specimen, but have the meaning of “at least a single specimen” and do not exclude a plurality. Expressions such as: “means for . . . ” should be read as: “component configured for . . . ” or “element constructed to . . . ” and should be construed to encompass all equivalents for the structures disclosed. The use of expressions like: “critical” “advantageous”, “preferably”, “desired”, etc. is not intended to limit the invention. Moreover, features that are not specifically or expressly described or claimed in the construction according to the invention but which are within the reach of the skilled person may also be encompassed without departing from the scope of protection as determined by the claims.

1. A method for determining calving information about a moment at which a cow is going to calve and/or a period in which a cow is going to calve, and/or the moment at which the cow has calved, wherein use is made of a sensor and a signal processing unit which is configured to process signals obtained with the sensor, with the aid of the sensor a heartbeat of the cow is measured so that the signals obtained with the sensor comprise information about the measured heartbeat of the animal, with the signal processing unit an increased heartbeat is recognized according to at least one predetermined criterion on the basis of signals obtained with the sensor and, if the increased heartbeat is recognized with the signal processing unit, the signal processing unit generates an alarm signal which comprises said calving information.

2. The method according to claim 1, wherein with the signal processing unit it is recognized that the measured heartbeat is greater than a first predetermined value.

3. The method according to claim 2, wherein:
the first predetermined value is equal to an average heartbeat of the cow over a predetermined period in the past plus a first increment, the first predetermined value is determined with the aid of the sensor and the signal processing unit; or
the first predetermined value is determined for a group of animals and is inputted at the signal processing unit.

4. (canceled)

5. (canceled)

6. The method according to claim 2, wherein with the signal processing unit it is recognized that the measured heartbeat is greater than a second predetermined value which is greater than the first predetermined value, for example wherein the second predetermined value is equal to the first predetermined value plus a second increment.

7. (canceled)

8. The method according to claim 2, wherein with the aid of the signal processing unit a first alarm signal is generated when the heartbeat is greater than the first predetermined value,
the first alarm signal indicates that the cow is going to calve within a predetermined first period, and
the time duration of the first period is predetermined.

9. The method according to claim 6, with the aid of the signal processing unit a second alarm signal is generated when the heartbeat is greater than the second predetermined value, the second signal indicates that the cow is going to calve within a predetermined second period, the time duration of the first and second period is predetermined and the second period has a shorter time duration than the first period.

10. The method according to claim 1, wherein with the aid of the signal processing unit a third alarm signal is generated when the cow has calved.

11. The method according to claim 10, wherein with the aid of the signal processing unit the third alarm signal is generated when it is detected that the heartbeat, after having risen substantially by more than a predetermined percentage of an instantaneous heartbeat of the animal such as 10%, 20% or 30%), has fallen again.

12. The method according to claim 2, wherein with the aid of the signal processing unit a third alarm signal is generated when it is detected by the signal processing unit that the heartbeat, after having exceeded the first predetermined value, has fallen below the first predetermined value again.

13. The method according to claim 6, wherein with the aid of the signal processing unit a third alarm signal is generated when it is detected by the signal processing unit that the heartbeat, after having exceeded the second predetermined value, has fallen below the second predetermined value again.

14. The method according to claim 1, wherein with the signal processing unit a fourth alarm signal is generated when in a third period, which begins at the moment that the heartbeat has risen substantially by more than a predetermined percentage of an instantaneous heartbeat of the animal such as 10%, 20% or 30%, and it is not detected that the heartbeat has fallen again.

15. The method according to claim 2, wherein with the signal processing unit a fourth alarm signal is generated when in a third period which begins at the moment that the heartbeat has exceeded the first predetermined value it is not detected by the signal processing unit that the heartbeat has fallen below the first predetermined value again and/or does not rise substantially further.

16. The method according to claim 6, wherein with the signal processing unit a fourth alarm signal is generated when in a third period which begins at the moment that the heartbeat has exceeded the second predetermined value it is not detected by the signal processing unit that the heartbeat has fallen below the second predetermined value again and/or does not rise substantially further.

17. (canceled)

18. (canceled)

19. (canceled)

20. The method according to claim 8, wherein the first period lasts 1-4 days.

21. The method according to claim 9, wherein the second period lasts 1-1440 minutes.

22. (canceled)

23. A system for determining calving information about a moment at which a cow is going to calve and/or a period in which a cow is going to calve and/or the moment at which the cow has calved, wherein the system comprises a sensor and a signal processing unit which is configured to process signals obtained with the sensor, the sensor is configured to measure a heartbeat of the cow so that the signals obtained with the sensor comprise information about the measured heartbeat of the animal and the signal processing unit is configured to recognize an increased heartbeat according to a predetermined criterion on the basis of signals obtained with the sensor and wherein the signal processing unit is configured for, if the increased heartbeat is recognized with the signal processing unit, generating an alarm signal which comprises said calving information.

24. The system according to claim 23, wherein the signal processing unit is configured to recognize that the measured heartbeat is greater than a first predetermined value, the first predetermined value is equal to an average heartbeat of the cow over a predetermined period in the past plus a first increment, the first predetermined value can be determined with the aid of the sensor and the signal processing unit; or the first predetermined value is determined for a group of animals and is inputted at the signal processing unit.

25. (canceled)

26. (canceled)

27. (canceled)

28. The system according to claim 24, wherein the signal processing unit is configured to recognize that the measured heartbeat is greater than a first predetermined value, and in that the signal processing unit is further configured to recognize that the measured heartbeat is greater than a second predetermined value which is greater than the first predetermined value the second predetermined value is equal to the first predetermined value plus a second increment.

29. (canceled)

30. The system according to claim 24, wherein the signal processing unit is configured to recognize that the measured heartbeat is greater than a first predetermined value, and that the signal processing unit is configured to generate a first alarm signal when the heartbeat is greater than the first predetermined value, wherein the first alarm signal indicates that the cow is going to calve within a predetermined first period, wherein the time duration of the first period is predetermined.

31. The system according to claim 28, wherein the signal processing unit is configured to generate a second alarm signal when the heartbeat is greater than the second predetermined value, the second alarm signal indicates that the cow is going to calve within a predetermined second period, the time duration of the second period is predetermined and the second period has a shorter time duration than the first period.

32. The system according to claim 23, wherein the signal processing unit is configured to generate a third alarm signal when the cow has calved, when it is detected by the signal processing unit that the heartbeat after having risen substantially by more than a predetermined percentage of an instantaneous heartbeat of the animal such as 10%, 20% or 30% and has fallen again and/or, when it is detected by the signal processing unit that the heartbeat after having exceeded the first predetermined value has fallen below the first predetermined value again and/or, when it is detected by the signal processing unit that the heartbeat after having exceeded the second predetermined value has fallen below the second predetermined value again.

33. The system according to claim 23, wherein the signal processing unit is configured to generate a fourth alarm signal when in a third period, which begins at the moment that the heartbeat has risen substantially by more than a predetermined percentage of an instantaneous heartbeat of the animal such as 10%, 20% or 30%), and it is not detected that the heartbeat has fallen again.

34. The system according to claim 24, wherein the signal processing unit is configured to generate a fourth alarm signal when in a third period which begins at the moment that the heartbeat has exceeded the first predetermined value it is not detected by the signal processing unit that the heartbeat has fallen below the first predetermined value again and/or does not rise substantially further.

35. The system according to claim 28, wherein the signal processing unit is configured to generate a fourth alarm signal when in a third period, which begins at the moment that the heartbeat has exceeded the second predetermined value, it is not detected by the signal processing unit that the heartbeat has fallen below the second predetermined value again and/or does not rise substantially further.

36. The system according to claim 30, wherein the system is configured to separate the animal by opening a gate so that the cow can enter a separation space, and closing the gate again, when the first alarm signal is delivered.

37. The system according to claim 30, wherein the system is configured to automatically send a signal to a farmer, on his mobile phone, when an alarm signal is delivered.

38. (canceled)

39. (canceled)

40. The system according to claim 23, wherein the sensor is selected from a group consisting of
a microphone, or
a system having a first unit to send light to the tissue of the animal and a second unit to receive light from the tissue, wherein the received light has propagated through the tissue of the animal and/or has reflected thereon, or
a blood pressure sensor, or
a video camera.

41. The system according to claim 23, wherein
the sensor is configured to communicate wirelessly with the signal processing unit; or the sensor is included in a tag which can be worn by the animal, a bolus which can be included in one of the stomachs of the animal or in an implantable element; or the sensor and the signal processing unit are included in a same housing; or the signal processing unit is configured to communicate wirelessly with a central computer to pass on an alarm signal generated by the signal processing unit to the central computer.

42. (canceled)

43. (canceled)

44. (canceled)

45. The system according to claim 23, wherein the sensor and the signal processing unit are included in a same housing and the housing is implemented as either a smart tag configured to be attached to the ear of an animal which can be worn by the animal, or a bolus which can be included in the stomach of the animal or in an implantable element; or the smart tag being configured to communicate wirelessly with the signal processing unit.

46. (canceled)

47. (canceled)

48. The system according to claim 23, wherein the sensor comprises a first unit to send light to the tissue of the animal and a second unit to receive light from the tissue, wherein the received light has propagated through the tissue of the animal and/or has reflected thereon.

49. The system according to claim 48, wherein the smart tag is implanted as an ear stud which comprises a first ear stud part and a second ear stud part which in use are respectively attached on either side of the ear, the first unit is included in the first part of the ear stud and the second unit is included in the second part of the ear stud.
